(19) United States
(12) Patent Application Publication
(10) Pub. No.: US 2003/0092770 A1
(43) Pub. Date: May 15, 2003

(54) Combination therapy for treatment of depression

(76) Inventor: Phil Skolnick, Edgewater, NJ (US)

Correspondence Address: ELI LILLY AND COMPANY, PATENT DIVISION, P.O. BOX 6288, INDIANAPOLIS, IN, 46206-6288, US

(21) Appl. No.: 10/258,558

(22) PCT Filed: May 11, 2001
(86) PCT No.: PCT/US01/11768

(51) Int. Cl. A61K 31/18; A61K 31/137

(52) U.S. Cl. 514/605; 514/649

(57) ABSTRACT

The present invention provides a method for treating depression, comprising administering to a patient an effective amount of a first component which is a suitable antidepressant, in combination with an effective amount of a second component which is a suitable AMPA receptor potentiator.

In the mammalian central nervous system (CNS), the transmission of nerve impulses is controlled by the interaction between a neurotransmitter, that is released by a sending neuron, and a surface receptor on a receiving neuron, which causes either an excitation or inhibition of this receiving neuron. L-Glutamate, which is the most abundant neurotransmitter in the CNS, mediates the majority of excitatory transmission in mammals, and is referred to as an excitatory amino acid (EAA). The receptors that respond to glutamate are generally referred to as excitatory amino acid receptors (EAA receptors). See Watkins & Evans, Ann. Rev. Pharmacol. Toxicol., 21, 165 (1981); Monaghan, Bridges, and Cotman, Ann. Rev. Pharmacol. Toxicol., 29, 365 (1989); Watkins, Krogsgaard-Larsen, and Honore, Trans. Pharm. Sci., 11, 25 (1990). The excitatory amino acids are of great physiological importance, playing a role in a variety of physiological processes, such as long-term potentiation (learning and memory), the development of synaptic plasticity, motor control, respiration, cardiovascular regulation, and sensory perception.

Excitatory amino acid receptors are classified into two general types.

Receptors that are directly coupled to the opening of cation channels in the cell membrane of the neurons are termed “ionotropic”. This type of receptor has been subdivided into at least three subtypes, which are defined by the selective agonists N-methyl-D-aspartate (NMDA), alpha-amino-3-hydroxy-5-methylisoxazole-4-propionic acid (AMPA), and kainic acid (KA). The second general type of receptor is the G-protein or second messenger-linked “metabotropic” excitatory amino acid receptor. This second type of EAA receptor is coupled to multiple second messenger systems that lead to enhanced phosphoinositide hydrolysis, activation of phospholipase D, increases or decreases in c-AMP formation, and changes in ion channel function. Schoepp and Conn, Trends in Pharmacol. Sci., 14, 13 (1993). Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

The AMPA subtype of glutamate receptors are assembled from four protein subunits known as GluR1 to GluR4, (also referred to as GluRA-GluRD) while kainic acid receptors are assembled from the subunits GluR5 to GluR7, and KA-1 and KA-2. Wong and Mayer, Molecular Pharmacology 44: 505-510, 1993. It is not yet known how these sub-units are combined in the natural state. However, the structures of certain human variants of each subunit have been elucidated, and cell lines expressing individual subunit variants have been cloned and incorporated into test systems designed to identify compounds which bind to or interact with them, and hence which may modulate their function. Thus, European patent application, publication number EP-A2-0574257 discloses the human sub-unit variants GluR1B, GluR2B, GluR3A and GluR3B. European patent application, publication number EP-A1-0583917 discloses the human subunit variant GluR4B.

One distinctive property of AMPA and kainic acid receptors is their rapid deactivation and desensitization in response to application of glutamate. Yamada and Tang, The Journal of Neuroscience, September 1993, 13(9): 3904-3915 and Kathryn M. Partin, J. Neuroscience, Nov. 1, 1996, 16(21): 6634-6647. The physiological implications of rapid desensitization, and deactivation if any, are not fully understood.

It is known that the rapid desensitization and deactivation of AMPA and/or kainic acid receptors to glutamate may be inhibited using certain compounds.

This action of these compounds is often referred to in the alternative as “potentiation” of the receptors. One such compound, which selectively potentiates AMPA receptor function, is cyclothiazide. Partin et al., Neuron. Vol. 11, 1069-1082, 1993.

In addition, certain sulfonamide derivatives which potentiate glutamate receptor function in a mammal have been disclosed in the following International Patent Application Publications: WO 98/33496 published Aug. 6, 1998; WO 99/43285 published Sep. 2, 1999; and WO 00/06539; WO 00/06537, WO 00/06176, WO 00/06159, WO 00/06158, WO 00/06157, WO 00/06156, WO 00/06149, WO 00/06148, and WO 00/06083, all published Feb. 10, 2000. Furthermore, International Patent Application Publication WO 97/39750, published Oct. 30, 1997 discloses a method of treating depression in a human suffering from a mental disorder comprising the selective potentiation of AMPA brain receptors of the patient to natural ligands thereof, the selective potentiation being sufficient to amplify the effects of the natural ligands in an amount adequate to improve the depression.

Depression in its many variations has recently become much more visible to the general public than it has previously been. It is now recognized as an extremely damaging disorder, and one that afflicts a surprisingly large fraction of the population. Suicide is the most extreme symptom of depression, but millions of people, not quite so drastically afflicted, live in misery and partial or complete uselessness, and afflict their families as well by their affliction. The introduction of fluoxetine, a serotonin reuptake inhibitor (SRI), was a breakthrough in the treatment of depression, and depressives are now much more likely to be diagnosed and treated than they were only a decade ago.

Depression is often associated with other diseases and conditions, or caused by such other conditions. For example, it is associated with Parkinson's disease; with HIV; with Alzheimer's disease; and with abuse of anabolic steroids. Depression may also be associated with abuse of any substance, or may be associated with behavioral problems resulting from or occurring in combination with head injuries, mental retardation or stroke.

Despite the breakthrough nature of serotonin reuptake inhibitors in the treatment of depression, a number of patients suffering from depression do not respond, or respond only partially to treatment with serotonin reuptake inhibitors, for example, or other traditional modes of treating depression, including the older tricyclic class of compounds referred to as monoamine oxidase inhibitors (MAOI's). Additionally, there is often a significant period of time before treatment with serotonin reuptake inhibitors provide a therapeutic effect. Furthermore, various side effects are sometimes associated with current antidepressant therapy, for example with serotonin reuptake inhibitors the gastrointestinal system may be affected wherein symptoms are often manifested as nausea and occasional vomiting. An additional troubling side effect associated with serotonin reuptake inhibitors is sexual dysfunction. It has been estimated that such sexual dysfunction is as high as 34%. [See F. M. Jacobsen, J. Clin. Psychiatry, 53, 119, (1992)]. These side effects often result in depressed patients not maintaining the SRI therapy for a period that is long enough in duration to recognize any significant improvement in the patient's condition.

The present invention provides a method for treating depression, comprising administering to a patient an effective amount of a first component which is a suitable antidepressant, in combination with an effective amount of a second component which is a suitable AMPA receptor potentiator.

The present invention further provides a method for treating refractory depression, comprising administering to a patient an effective amount of a first component which is a suitable antidepressant, in combination with an effective amount of a second component which is a suitable AMPA receptor potentiator.

This invention provides, further, a method for attenuating adverse events associated with depression comprising administering to a patient an effective amount of a first component which is a suitable antidepressant, in combination with an effective amount of a suitable AMPA receptor potentiator.

This invention also provides a method of providing rapid onset treatment of depression to a patient which comprises administering to said patient an effective amount of a first component which is a suitable antidepressant, in combination with an effective amount of a second component which is a suitable AMPA receptor potentiator.

The invention also provides a pharmaceutical composition which comprises a first component which is a suitable antidepressant, and a second component which is a suitable AMPA receptor potentiator, the two components being present in an amount effective in the treatment of depression.

The present invention further provides an article of manufacture comprising packaging material and a pharmaceutical composition which comprises a first component which is a suitable antidepressant, and a second component which is a suitable AMPA receptor potentiator, contained within said packaging material, wherein said packaging material comprises a label which indicates that said pharmaceutical composition can be used for treating depression.

### BRIEF DESCRIPTION OF THE DRAWING

FIG. 1 discloses the effect of the combination of imipramine and N-2-(4-(3-thienyl)phenylpropyl 2-propanesulfonamide (392098) in the Forced Swim Test in the mouse. More specifically, low doses of 392098 by itself produced no effect in the Forced Swim Test. FIG. 1 further reveals that when 392098 was combined at a dose as low as 25 micrograms/kg with a subeffective dose of imipramine (5 mg/kg), a statistically significant reduction in immobility of the mouse resulted.

### DETAILED DESCRIPTION OF THE INVENTION

As used herein the term “potentiating glutamate receptor function” refers to any increased responsiveness of glutamate receptors, for example AMPA receptors, to glutamate or an agonist, and includes but is not limited to inhibition of rapid desensitization or deactivation of AMPA receptors to glutamate.

As used herein the term “AMPA receptor potentiator” refers to a compound which inhibits the rapid desensitization or deactivation of AMPA receptors to glutamate.

As used herein the term “atenuating” means decreasing the number, severity or frequency of side effects or adverse events associated with treatment of depression with conventional antidepressant medication, such as an SRI's, when such products are used at dosages that yield beneficial effects on the symptoms of the disease.

As used herein the number “392098” refers to the compound N-2-(4-(3-thienyl)phenylpropyl 2-propanesulfonamide of example 2.

As used herein the term “IMI” refers to imipramine.

As used herein the term “FST” refers to Forced Swim Test.

As used herein the term “i.p.” refers to intraperitoneal or intraperitoneally.

It is understood by one of ordinary skill in the art that the present invention includes the pharmaceutically acceptable salts of either or both of the first and second components. The compounds used in this invention can possess a sufficiently acidic group, a sufficiently basic group, or both functional groups, and accordingly react with any of a number of organic and inorganic bases, and inorganic and organic acids, to form a pharmaceutically acceptable salt.

The term “pharmaceutically acceptable salt” as used herein, refers to salts of the compounds used in the present invention which are substantially non-toxic to living organisms. Typical pharmaceutically acceptable salts include those salts prepared by reaction of the compounds of the present invention with a pharmaceutically acceptable mineral or organic acid. Such salts are also known as acid addition salts. Such salts include the pharmaceutically acceptable salts listed in Journal of Pharmaceutical Science, 66, 2-19 (1977) which are known to the skilled artisan.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprate, caprylate, acrylate, ascorbate, formate, hydrochloride, dihydrochloride, isobutyrate, caproate, heptanoate, propiolate, propionate, phenylpropionate, salicylate, oxalate, malonate, succinate, suberate, sebacate, fumarate, malate, maleate, hydroxymaleate, mandelate, nicotinate, isonicotinate, cinnamate, hippurate, nitrate, phthalate, teraphthalate, butyne-1,4-dioate, butyne-1,4-dicarboxylate, hexyne-1,4-dicarboxylate, hexyne-1,6-dioate, benzoate, chlorobenzoate, methylbenzoate, hydroxybenzoate, methoxybenzoate, dinitrobenzoate, o-acetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, α-hydroxybutyrate, glycolate, tartrate, benzenesulfonate, methanesulfonate, ethanesulfonate, propanesulfonate, hydroxyethanesulfonate, naphthalene-1-sulfonate, napththalene-2-sulfonate, mandelate, tartarate, and the like. Preferred pharmaceutically acceptable acid addition salts are those formed with mineral acids such as hydrochloric acid and hydrobromic acid, and those formed with organic acids such as maleic acid, oxalic acid and methanesulfonic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred.

It should be recognized that the particular counterion forming a part of any salt of this invention is usually not of a critical nature, so long as the salt as a whole is pharmacologically acceptable and as long as the counterion does not contribute undesired qualities to the salt as a whole. It is further understood that the above salts may form hydrates or exist in a substantially anhydrous form.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ee = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 70:30 is achieved, the ee with respect to the first enantiomer is 40%. However, if the final ratio is 90:10, the ee with respect to the first enantiomer is 80%. An ee of greater than 90% is preferred, an ee of greater than 95% is most preferred and an ee of greater than 99% is most especially preferred.

Enantiomeric enrichment is readily determined by one of ordinary skill in the art using standard techniques and procedures, such as gas or high performance liquid chromatography with a chiral column. In addition, separation and isolation of the compounds of the present invention into the individual enantiomers is similarly performed by one of ordinary skill in the art using standard techniques and procedures, such as gas or high performance liquid chromatography with a chiral column, or other standard resolving techniques. Choice of the appropriate chiral column, eluent and conditions necessary to effect separation of the enantiomeric pair is well within the knowledge of one of ordinary skill in the art. In addition, the enantiomers of compounds of the present invention can be resolved using standard techniques such as those described by J. Jacques, et al., “Enantiomers, Racemates, and Resolutions”, John Wiley and Sons, Inc., 1981, and E. L. Eliel and S. H. Wilen, “Stereochemistry of Organic Compounds”, (Wiley-Interscience 1994), and European Patent Application No. EP-A-838448, published Apr. 29, 1998.

Some of the compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

The designation “ [symbol] ” refers to a bond that protrudes forward out of the plane of the page.

The designation “ [symbol] ” refers to a bond that protrudes backward out of the plane of the page.

The designation “ [symbol] ” refers to a bond wherein the stereochemistry is not defined.

As used herein the term “CX516” refers to a compound of the following structure:

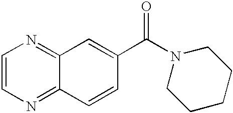

The first component is a compound which functions as a suitable antidepressant. As used herein the term “suitable antidepressant” includes but is not limited to serotonin reuptake inhibitors (SRI's), norepinephrine reuptake inhibitors (NERI's), combined serotonin-norepinephrine reuptake inhibitors (SNRI's), monoamine oxidase inhibitors (MAOI's), phosphodiesterase-4 inhibitors (PDE4), and the like. SRI's and SNRI's are the preferred suitable antidepressants, with SRI's being most preferred.

More specifically, examples of suitable antidepressants include, but are not limited to:

Fluoxetine, N-methyl-3-(p-trifluoromethylphenoxy)-3-phenylpropylamine, is marketed in the hydrochloride salt form, and as the racemic mixture of its two enantiomers. U.S. Pat. No. 4,314,081 is an early reference on the compound. Robertson et al., J. Med. Chem. 31, 1412 (1988), taught the separation of the R and S enantiomers of fluoxetine and showed that their activity as serotonin uptake inhibitors is similar to each other. In this document, the word “fluoxetine” will be used to mean any acid addition salt or the free base, and to include either the racemic mixture or either of the R and S enantiomers;

Duloxetine, N-methyl-3-(1-naphthalenyloxy)-3-(2-thienyl)propanamine, is usually administered as the hydrochloride salt and as the (+) enantiomer. It was first taught by U.S. Pat. No. 4,956,388, which shows its high potency. The word “duloxetine” will be used here to refer to any acid addition salt or the free base of the molecule;

Venlafaxine is known in the literature, and its method of synthesis and its activity as an inhibitor of serotonin and norepinephrine uptake are taught by U.S. Pat. No. 4,761,501. Venlafaxine is identified as compound A in that patent;

Milnacipran (N,N-diethyl-2-aminomethyl-1-phenylcyclopropanecarboxamide) is taught by U.S. Pat. No. 4,478,836, which prepared milnacipran as its Example 4. The patent describes its compounds as antidepressants. Moret et al., Neuropharmacology 24, 1211-19 (1985), describe its pharmacological activities as an inhibitor of serotonin and norepinephrine reuptake;

Citalopram, 1-[3-(dimethylamino)propyl]-(4-fluorophenyl)-1,3-dihydro-5-isobenzofurancarbonitrile, is disclosed in U.S. Pat. No. 4,136,193 as a serotonin reuptake inhibitor. Its pharmacology was disclosed by Christensen et al., Eur. J. Pharmacol. 41, 153 (1977), and reports of its clinical effectiveness in depression may be found in Dufour et al., Int. Clin. Psychopharmacol. 2, 225 (1987), and Timmerman et al., ibid., 239;

Fluvoxamine, 5-methoxy-1-[4-(trifluoromethyl)phenyl]-1-pentanone O-(2-aminoethyl)oxime, is taught by U.S. Pat. No. 4,085,225. Scientific articles about the drug have been published by Claassen et al., Brit. J. Pharmacol. 60, 505 (1977); and De Wilde et al., J. Affective Disord. 4, 249 (1982); and Benfield et al., Drugs 32, 313 (1986);

Paroxetine, trans-(−)-3-[(1,3-benzodioxol-5-yloxy)methyl]-4-(4-fluorophenyl)piperidine, may be found in U.S. Pat. Nos. 3,912,743 and 4,007,196. Reports of the drug's activity are in Lassen, Eur. J. Pharmacol. 47, 351 (1978); Hassan et al., Brit. J. Clin. Pharmacol. 19, 705 (1985); Laursen et al., Acta Psychiat. Scand. 71, 249 (1985); and Battegay et al., Neuropsychobiology 13, 31 (1985);

Sertraline, (1S-cis)-4-(3,4-dichlorophenyl)-1,2,3,4-tetrahydro-N-methyl-1-naphthylamine hydrochloride, is a serotonin reuptake inhibitor which is marketed as an antidepressant. It is disclosed by U.S. Pat. No. 4,536,518;

Buproprion (Welibutrin®), (±)-1-(3-chlorophenyl)-2-[(1,1-dimethylethyl)amino]-1-propanone is indicated for treatment of depression. The term “buproprion” “will be used here to refer to any acid addition salt or the free base of the molecule existing as the racemate or either enantiomer; The HCl salt is particularly preferred;

Reboxetine (Edronax™), 2-[α-(2-ethoxy)phenoxy-benzyl]morpholine, is usually administered as the racemate. It was first taught by U.S. Pat. No. 4,229,449, which describes its utility for the treatment of depression. Reboxetine is a selective norepinephrine reuptake inhibitor. The term “reboxetine” will be used here to refer to any acid addition salt or the free base of the molecule existing as the racemate or either enantiomer;

Moclobemide, 4-chloro-N-[2-(4-morpholinyl)-ethyl]benzamide, see U.S. Pat. No. 4,210,754;

Imipramine, see U.S. Pat. No. 2,554,736; and

Rolipram, see U.S. Pat. No. 4,193,926.

The second component is a compound which is a suitable AMPA potentiator. Examples of suitable AMPA receptor potentiators include but are not limited to those disclosed in:

WO 98/33496 published Aug. 6, 1998;

WO 99/43285 published Sep. 2, 1999;

WO 00/06539 published Feb. 10, 2000;

WO 00/06537 published Feb. 10, 2000;

WO 00/06176 published Feb. 10, 2000;

WO 00/06159 published Feb. 10, 2000;

WO 00/06158 published Feb. 10, 2000;

WO 00/06157 published Feb. 10, 2000;

WO 00/06156 published Feb. 10, 2000;

WO 00/06149 published Feb. 10, 2000;

WO 00/06148 published Feb. 10, 2000;

WO 00/06083 published Feb. 10, 2000;

U.S. Pat. No. 5,891,871, issued Apr. 6, 1999;

U.S. Pat. No. 5,852,008, issued Dec. 22, 1998;

U.S. Pat. No. 5,747,492, issued May 5, 1998; and

U.S. Pat. No. 5,650,409, issued Jul. 22, 1997; the disclosures of which are all hereby incorporated by reference. The above suitable AMPA receptor potentiators are readily prepared by one of ordinary skill in the art following, for example, the procedures set forth therein.

Specific examples of suitable AMPA receptor potentiators are listed in table I.

TABLE I
Suitable AMPA Receptor Potentiators

| Example | Compound |
|---|---|
| 1 | [(Methylethyl)sulfonyl]{2-[4-(4-{2- |
| | [(methylsulfonyl)amino]ethyl}phenyl)phenyl]propyl}a |
| | mine |
| 1a | {(2R)-2-[4-(4-{2- |
| | [(Methylsulfonyl)amino]ethyl}phenyl)phenyl]propyl} |
| | [(methylethyl)sulfonyl]amine |
| 2 | N-2-(4-(3-thienyl)phenylpropyl 2- |
| | propanesulfonamide |
| 3 | [2-fluoro-2-(4-{3- |
| | [(methylsulfonyl)amino]phenyl}phenyl)propyl][(meth |
| | ylethyl)sulfonyl]amine |
| 3a | [2-fluoro-2-(4-{3- |
| | [(methylsulfonyl)amino]phenyl}phenyl)propyl][(meth |
| | ylethyl)sulfonyl]amine (enantiomer 1) |
| 4 | CX516 |
| 5 | Aniracetam |
| 6 | Piracetam |
| 7 | PEPA |
| 8 | IDRA-21 |
| 9 | S18986 |

All of the U.S. patents which have been mentioned above in connection with compounds used in the present invention are incorporated herein by reference.

While all combinations of first and second components are useful and valuable, certain combinations are particularly valued and are preferred, as set forth in table II:

TABLE II
Particularly Preferred Combinations.

| First Component | Second Component |
|---|---|
| Fluoxetine | Suitable AMPA receptor potentiator |
| Venlafaxine | Suitable AMPA receptor potentiator |
| Citralopram | Suitable AMPA receptor potentiator |
| Fluvoxamine | Suitable AMPA receptor potentiator |
| Paroxetine | Suitable AMPA receptor potentiator |
| Sertraline | Suitable AMPA receptor potentiator |
| Milnacipran | Suitable AMPA receptor potentiator |
| Duloxetine | Suitable AMPA receptor potentiator |
| Fluoxetine | [(Methylethyl)sulfonyl]{2-[4-(4-{2- |
| | [(methylsulfonyl)amino]ethyl}phenyl)phenyl]propyl}a |
| | mine |
| Fluoxetine | {(2R)-2-[4-(4-{2- |
| | [(Methylsulfonyl)amino]ethyl}phenyl)phenyl]propyl} |
| | [(methylethyl)sulfonyl]amine |
| Fluoxetine | CX516 |
| Paroxetine | {(2R)-2-[4-(4-{2- |
| | [(Methylsulfonyl)amino]ethyl}phenyl)phenyl]propyl} |
| | [(methylethyl)sulfonyl]amine |
| Paroxetine | CX516 |
| Citalopram | {(2R)-2-[4-(4-{2- |
| | [(Methylsulfonyl)amino]ethyl}phenyl)phenyl]propyl}[( |
| | methylethyl)sulfonyl]amine |
| Citalopram | CX516 |
| Sertraline | {(2R)-2-[4-(4-{2- |
| | [(Methylsulfonyl)amino]ethyl}phenyl)phenyl]propyl} |
| | [(methylethyl)sulfonyl]amine |
| Sertraline | CX516 |
| Imipramine | {(2R)-2-[4-(4-{2- |
| | ((Methylsulfonyl)amino]ethyl}phenyl)phenyl]propyl} |
| | [(methylethyl)sulfonyl]amine |
| Imipramine | CX516 |
| Imipramine | N-2-(4-(3-thienyl)phenylpropyl 2- |
| | propanesulfonamide |
| Imipramine | [2-fluoro-2-(4-{3- |
| | [(methylsulfonyl)amino]phenyl}phenyl)propyl] |
| | [(methylethyl)sulfonyl]amine (enantiomer 1) |

The following examples and preparations represent typical syntheses of certain AMPA receptor potentiators as described generally above. These examples are illustrative only and are not intended to limit the invention in any way. The reagents and starting materials are readily available to one of ordinary skill in the art. As used herein, the following terms have the meanings indicated: “eq” refers to equivalents; “g” refers to grams; “mg” refers to milligrams; “L” refers to liters; “mL” refers to milliliters; “μL” refers to microliters; “mol” refers to moles; “mmol” refers to millimoles; “psi” refers to pounds per square inch; “min” refers to minutes; “h” or “hr” refers to hours; “° C.” refers to degrees Celsius; “TLC” refers to thin layer chromatography; “HPLC” refers to high performance liquid chromatography; “Rf” refers to retention factor; “Rt” refers to retention time; “δ” refers to part per million down-field from tetramethylsilane; “THF” refers to tetrahydrofuran; “DMF” refers to N,N-dimethylformamide; “DMSO” refers to methyl sulfoxide; “LDA” refers to lithium diisopropylamide; “EtOAc” refers to ethyl acetate; “aq” refers to aqueous; “iPrOAc” refers to isopropyl acetate; “methyl DAST” refers to dimethylaminosulfur trifluoride, “DAST” refers to diethylaminosulfur trifluoride, “DBU” refers to 1,8-diazabicyclo[5.4.0]undec-7-ene; as used herein “Pd(dppf)₂Cl₂ catalyst” refers to ([1,1′-bis(diphenylphosphino)ferrocene]dichloropalladium(II) complex with CH₂Cl₂; as used herein the terms “Me”, “Et”, “Pr”, “iPr”, and “Bu” refer to methyl, ethyl, propyl, isopropyl, and butyl respectively, and “RT” refers to room temperature.

### EXAMPLE 1

Preparation of [(methylethyl)sulfonyl]{2-[4-(4-{2[(methylsulfonyl)amino]ethyl}phenyl)pheny]propyl}amine.

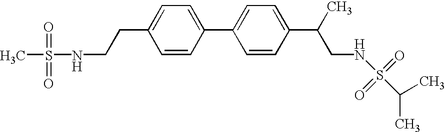

The title compound can be prepared following the procedure disclosed in WO 98/33496 published Aug. 6, 1998, Example 51). More specifically, to a room temperature solution of 0.1 g (0.3 mmol) of N-2-(4-(4-(2-aminoethyl)phenyl)phenyl)propyl 2-propanesulfonamide (prepared following procedure disclosed in WO 98/33496 published Aug. 6, 1998, Example 50) and 0.06 mL (0.4 mmol) of triethylamine in 2 mL of dichloromethane was added 0.03 mL (0.4 mmol) of methanesulfonyl chloride. The mixture was stirred at ambient temperature for 16 hours. Chromatography (10 g silica gel, 50% ethyl acetate/hexane) of the reaction mixture afforded 0.1 g (94%) of the title compound.

Analysis calculated for C₂₁H₃₀N₂O₄S₂:

Theory % C, 57.51; % H, 6.89; % N, 6.39.

Found: % C, 57.90; % H, 6.72; % N, 6.

### EXAMPLE 1a

Preparation of {(2R)-2-[4-(4-{2-[(methylsulfonyl)amino]ethyl}phenyl)phenyl]propyl}[(methylethyl)sulfony]amine.

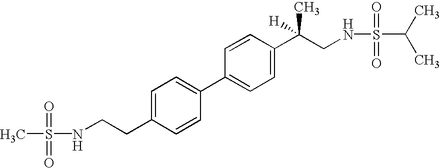

Preparation of 2-Phenyl-1-propylamine HCl.

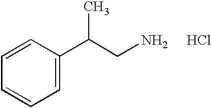

To an autoclave hydrogenation apparatus under nitrogen was charged water-wet 5% palladium on carbon (453 g), ethanol (6.36 L), 2-phenylpropionitrile (636 g, 4.85 moles) and finally concentrated (12M) hydrochloric acid (613 g, 5.6 mole). The mixture was stirred rapidly and pressurized to 75-78 psi with hydrogen. The mixture was then heated to 50-64° C. for 3 hours. ¹H NMR analysis of an aliquot showed less than 5% starting material. The reaction mixture was depressurized and filtered to afford two lots of filtrate that were concentrated under reduced pressure to ˜400 mL each. To each lot was added methyl tert-butyl ether (MTBE) (2.2 L each) and the precipitate solids were allowed to stir overnight. Each lot was filtered and the collected solids were each washed with fresh MTBE (100 mL) and dried overnight. The lots were combined to afford 2-phenyl-1-propylamine HCl (634.4 g, 76.2%) as a white powder.

1H NMR analysis of the free base: 1H NMR (CDCl3, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

Preparation of (2R)-2-phenylpropylamine Malate.

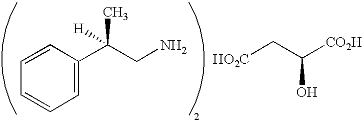

To a dry 3-Liter round bottom flask under nitrogen was charged 2-phenyl-1-propylamine HCl (317.2 g, 1.85 moles), dry ethanol (2.0 L) and NaOH beads (75.4 g, 1.89 moles) that were washed in with additional ethanol (500 mL). The mixture was stirred for 1.6 hours, and the resulting milky white NaCl salts were filtered. An aliquot of the filtrate was analyzed by gas chromatography to provide the amount of free amine, 2-phenyl-1-propylamine, (1.85 moles). A solution of L-malic acid (62.0 g, 0.462 mole, 0.25 equivalents) in ethanol (320 mL) was added dropwise to the yellow filtrate and the solution was heated to 75° C. The solution was stirred at 75° C. for 30 minutes. The heat was removed and the solution was allowed to cool slowly. The resulting thick precipitate was allowed to stir overnight. The precipitate was filtered and dried under vacuum after rinsing with ethanol (325 mL) to afford (2R)-2-phenylpropylamine malate (147.6 g, 39.5%) as a white crystalline solid. Chiral GC analysis of the free base, 2-phenyl-1-propylamine revealed 83.2% e.e. enriched in the R-isomer (configuration was assigned via spectrometric comparison with commercial 2-phenyl-1-propylamine) ¹H NMR (CDCl₃, 300 MHz) δ 7.32 (m, 2H), 7.21 (m, 3H), 2.86 (m, 2H), 2.75 (m, 1H), 1.25 (d, 3H, J=6.9), 1.02 (br s, 2H).

A slurry of (2R)-2-phenylpropylamine malate (147.1 g, 83.2% e.e.) in 1325 mL ethanol and 150 mL deionized water was heated to reflux (˜79.2° C.) until the solids went into solution. The homogeneous solution was allowed to slowly cool with stirring overnight. The precipitated white solids were cooled (0-5° C.) and filtered. The collected solids were rinsed with ethanol (150 mL) and dried at 35° C. to afford (2R)-2-phenylpropylamine malate (125.3 g, 85.2% recovery) as a white powder. Chiral GC analysis of the free base, (2R)-2-phenylpropylamine, revealed 96.7% e.e. enriched in the R-isomer.

¹H NMR (CD₃OD, 300 MHz) δ 7.32 (m, 10H), 4.26 (dd, 1H, J=3.6, 9.9), 3.08 (m, 6H), 2.72 (dd, 1H, J=9.3, 15.3), 2.38 (dd, 1H, J=9.3, 15.6), 1.33 (d, 6H, J=6.6).

Preparation of ((2R)-2-phenylpropyl)[(methylethyl)sulfonyl]amine.

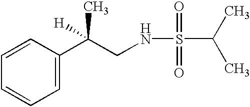

To a stirred slurry of (2R)-2-phenylpropylamine malate (200 g, 0.494 mol) in CH₂Cl₂ (1000 mL) was added 1.0 N NaOH (1050 mL, 1.05 moles). The mixture was stirred at room temperature for 1 hour and the organic phase was separated and gravity filtered into a 3.0 L round-bottom flask with a CH₂Cl₂ rinse (200 mL). The resulting free base, (2R)-2-phenylpropylamine, was dried via azeotropic distillation. Accordingly, the clear filtrate was concentrated to 600 mL at atmospheric pressure via distillation through a simple distillation head. Heptane (1000 mL) was added and the solution was concentrated again at atmospheric pressure to 600 mL using a nitrogen purge to increase the rate of distillation. The final pot temperature was 109° C.

The solution was cooled to room temperature under nitrogen with stirring to give a clear, colorless heptane solution (600 mL) of (2R)-2-phenylpropylamine. To this solution was added 4-dimethylaminopyridine (6.04 g, 0.0494 mol), triethylamine (200 g, 1.98 moles), and CH₂Cl₂ (500 mL). The mixture was stirred at room temperature until a clear solution was obtained. This solution was cooled to 5° C. and a solution of isopropylsulfonyl chloride (148 g, 1.04 moles) in CH₂Cl₂ (250 mL) was added dropwise with stirring over 2 hrs. The mixture was allowed to warm gradually to room temperature over 16 h. GC analysis indicated complete consumption of the (2R)-2-phenylpropylamine starting material.

The stirred mixture was cooled to 8° C. and 2 N HCl (500 mL) was added dropwise. The organic phase was separated and extracted with water (1×500 mL) and saturated NaHCO₃ (1×500 mL). The organic phase was isolated, dried (Na₂SO₄), and gravity filtered. The filtrate was concentrated under reduced pressure to provide ((2R)-2-phenylpropyl)[(methylethyl)sulfonyl]amine (230 g, 96%) as a pale yellow oil. ¹H NMR (CDCl₃, 300 MHz) δ 7.34 (m, 2H), 7.23 (m, 3H), 3.89 (br t, 1H, J=5.4), 3.36 (m, 1H), 3.22 (m, 1H), 3.05 (m, 1H), 2.98 (m, 1H), 1.30 (d, 3H, J=7.2), 1.29 (d, 3H, J-6.9), 1.25 (d, 3H, J=6.9).

Preparation of [(2R)-2-(4-iodophenyl)propyl][(methylethyl)sulfonyl]amine.

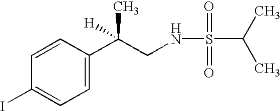

A stirred room temperature solution of ((2R)-2-phenylpropyl)[(methylethyl)sulfonyl]amine (37.1 g, 0.154 mol) in glacial acetic acid (185 mL) was treated with concentrated H₂SO₄ (16.0 g, 0.163 mol), added dropwise in a slow stream, followed by a H₂O rinse (37 mL). To this solution (˜30° C.) was added H₅lO₆ (8.29 g, 0.0369 mol), followed by iodine (17.9 g, 0.0707 mol). The resulting reaction mixture was heated and allowed to stir for 3 h at 60° C. After HPLC analysis verified the consumption of starting material, the reaction mixture was cooled to 30° C. and a 10% aqueous solution of NaHSO₃ (220 mL) was added dropwise while maintaining the temperature between 25° C. and 30° C. The mixture crystallized to a solid mass upon cooling to 0-5° C.

The solids were suction filtered and rinsed with H₂O to afford 61.7 g of crude solids that were redissolved into warm MTBE (500 mL). This solution was extracted with H₂O (2×200 mL) and saturated NaHCO₃ (1×200 mL) and the organic phase was dried (MgSO₄), filtered, and concentrated under reduced pressure to ˜200 mL. Heptane (100 mL) was added dropwise to the product solution with slow stirring until crystallization commenced. An additional 100 mL of heptane was added and the resulting suspension was allowed to stir slowly overnight at room temperature. The mixture was then cooled (0° C.), filtered, and the collected solids were rinsed with heptane. The solids were then air-dried to afford the intermediate title compound, [(2R)-2-(4-iodophenyl)propyl][(methylethyl)sulfonyl]amine (33.7 g, 59.8%) as a white powder. Chiral Chromatography of this lot indicated 100% e.e.

¹H NMR (CDCl₃, 300 MHz) δ 7.66 (d, 2H, J=8.1), 6.98 (d, 2H, J=8.4), 3.86 (br t, 1H, J=5.1), 3.33 (m, 1H), 3.18 (m, 1H), 3.06 (m, 1H), 2.92 (m, 1H), 1.30 (d, 3H, J=6.6), 1.27 (d, 6H, J=6.6).

Preparation of (methylsulfonyl)(2-phenylethyl)amine.

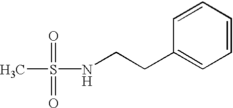

To a 10° C. solution of phenethylamine (12.1 g, 0.100 mol) and triethylamine (11.1 g, 0.110 mol) in CH₂Cl₂ (50 mL) was added methanesulfonyl chloride (12.6 g, 0.110 mol) dropwise over 10 min. The solution was stirred at room temperature for 1.5 h and was then washed with 1 N HCl (5×20 mL). The organic phase was directly concentrated to provide the intermediate title compound, (methylsulfonyl)(2-phenylethyl)amine, (21.2 g, 93.3%) as an oil.

¹H NMR (CDCl₃, 300 MHz) δ7.32 (m, 2H), 7.23 (m, 3H), 4.30 (br s, 1H), 3.40 (t, 2H, J=3.9), 2.88 (t, 2H, J=4.2), 2.81 (s, 3H).

Preparation of [2-(4-iodophenyl)ethyl](methylsulfonyl)amine.

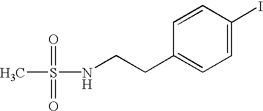

To a stirring room temperature solution of (methylsulfonyl)(2-phenylethyl)amine (205 g, 1.03 moles), water (200 mL), 95% sulfuric acid (111 g, 1.08 moles) in acetic acid (1 L), was added iodine (111 g, 0.438 mol) and periodic acid. (H₅IO₆, 45.6 g, 0.206 mol). The reaction mixture was warmed to 70-75° C. for 3 h. The heat was removed and the dark violet reaction mixture was allowed to proceed overnight at room temperature. Potassium hydroxide pellets (85%, 143 g, 2.16 moles) were added to neutralized the sulfuric acid and then enough saturated aqueous sodium sulfite was added to decolorize the mixture to afford a white suspension. The suspension was cooled to 15° C. and filtered. The filter cake was triturated thoroughly with water and was then dissolved in CH₂Cl₂ (1 L) and extracted with additional water (2×200 mL). The organic phase was concentrated under reduced pressure to provide the intermediate title compound, [2-(4-iodophenyl)ethyl](methylsulfonyl)amine, (201 g, 60.2%) as a white powder.

¹H NMR (CDCl₃, 300 MHz) δ 7.64 (d, 2H, J=4.8), 6.97 (d, 2H, J=5.1), 4.37 (br t, 1H, J=4), 3.36 (app. q, 2H, J=3.9), 2.85 (s, 3H), 2.82 (t, 2H, J=3.9).

Preparation of (tert-butoxy)-N-[2-(4-iodophenyl)ethyl]-N-(methylsulfonyl)carboxamide.

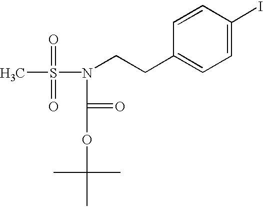

A room temperature solution of [2-(4-iodophenyl)ethyl](methylsulfonyl)amine (201 g, 0.618 mol), 4-dimethylaminopyridine (3.8 g, 0.031 mol) and di-tert-butyl dicarbonate (162 g, 0.744 mol) in CH₂Cl₂ (1 L) was allowed to stir overnight. The reaction mixture was washed with water (2×400 mL) and the organic phase was concentrated to about 600 mL and hexanes (400 mL) was added. This combined solution was washed again with water (400 mL) and was concentrated to a solid that was suspended in hexanes (600 mL) and filtered. The collected solids were dried under reduced pressure to afford the intermediate title compound, (tert-butoxy)-N-[2-(4-iodophenyl)ethyl]-N-(methylsulfonyl)carboxamide (241.5 g, 91.5%) as a white solid.

¹H NMR (CDCl₃, 300 MHz) δ 7.63 (d, 2H, J=7.8), 6.98 (d, 2H, J=7.8), 3.88 (t, 2H, J=6.9), 3.10 (s, 3H), 2.88 (t, 2H, J=6.9), 1.51 (s, 9H).

Preparation of (tert-butoxy)-N-(methylsulfonyl)-N-{2-[4-(44.5,5-tetramethyl(1,3,2-dioxaborolan-2-yl))phenyl]ethyl}carboxamide.

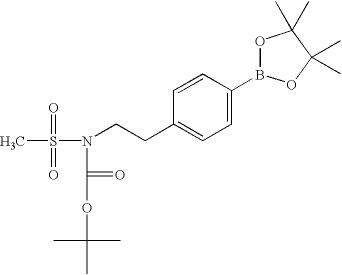

To a degassed solution of (tert-butoxy)-N-[2-(4-iodophenyl)ethyl]-N-(methylsulfonyl)carboxamide (128 g, 0.300 mol), triethylamine (91.1 g, 0.900 mol), and 1,1′-bis(diphenylphosphino) ferrocenedichloropalladium (II)—CH₂Cl₂ complex (2.9 g, 0.0035 mol) in acetonitrile (600 mL) was added pinacolborane (50 g, 0.391 mol) dropwise. The mixture was stirred at 70-74° C. for 8 h and then was cooled to room temperature. The reaction mixture was concentrated to a fluid oil that was partitioned between MTBE (500 mL) and water (500 mL). The organic phase was separated and washed with water (2×200 mL) and concentrated to a residue that was partially dissolved with heptane (1 L). The heptane soluble fraction was filtered through Celite® 521 and concentrated to an oil (95 g). The residue was dissolved in acetone (600 mL) and heptane (600 mL) and filtered through Celite® 521. The combined filtrates were concentrated to 95 g of a mixture of a 3:1 molar ratio (¹H NMR, 81.0% by weight) of intermediate title compound, (tert-butoxy)-N-(methylsulfonyl)-N-{2-[4-(4,4,5,5-tetramethyl(1,3,2-dioxaborolan-2-yl))phenyl]ethyl}carboxamide, (60.3% potency corrected yield) and protio derivative.

¹H NMR (CDCl₃, 300 MHz) δ 7.75 (d, 2H, J=7.8), 7.23 (d, 2H, J=8.1), 3.87 (t, 2H, J=8.1), 2.99 (s, 3H), 2.90 (t, 2H, J=7.5), 1.53 (s, 9H), 1.33 (s, 6H), 1.27 (s, 6H).

Preparation of (methylsulfonyl){2-[4-(4,4,5,5-tetramethyl(1,3,2-dioxaborolan-2-yl))phenyl]ethyl}amine.

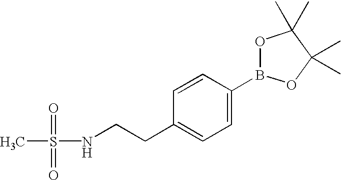

To a 2 L flask charged with a stirring solution of (tert-butoxy)-N-(methylsulfonyl)-N-{2-[4-(4,4,5,5-tetramethyl(1,3,2-dioxaborolan-2-yl))phenyl]ethyl}carboxamide (98.7 g, 0.232 mol) in CH₂Cl₂ (500 mL) was added trifluoroacetic acid (82 mL, 121.4 g, 1.06 moles) dropwise from an addition funnel. No exotherm was observed and the reaction solution was allowed to stir at room temperature for 18 h.

HPLC analysis indicated 98% completion so the cooled (5° C.) reaction mixture was neutralized by the slow addition of 5N NaOH (175 mL). The pH of the aqueous phase was 10.5. The phases were separated and the aqueous phase was extracted with CH₂Cl₂ (50 mL). The combined CH₂Cl₂ phases were washed with brine (2×100 mL) and water (1×100 mL). The CH₂Cl₂ phase was diluted with heptane (300 mL) and was concentrated under reduced pressure to afford a suspension that was isolated by filtration. The collected solids were washed with pentane (2×100 mL) and dried under vacuum to provide the intermediate title compound, (methylsulfonyl){2-[4-(4,4,5,5-tetramethyl(1,3,2-dioxaborolan-2-yl))phenyl]ethyl}amine, (69.0 g, 91.4%) as a white powder.

¹H NMR (CDCl₃, 300 MHz) δ 7.77 (d, 2H, J=8.1), 7.22 (d, 2H, J=7.8), 4.26 (br t, 1H, J=6), 3.40 (q, 2H, J=6.9), 2.89 (t, 2H, J=6.6), 2.82 (s, 3H), 1.34 (s, 12H).

Preparation of 4-{2-[(methylsulfonyl)amino]ethyl}benzene Boronic Acid.

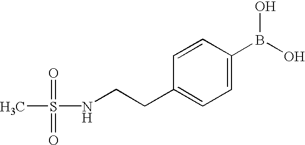

(Methylsulfonyl){2-[4-(4,4,5,5-tetramethyl(1,3,2-dioxaborolan-2-yl))phenyl]ethyl}amine (68.0 g, 0.209 mol) was placed into a 2L flask and combined with acetone (600 mL), 1N ammonium acetate (600 mL), and NaIO₄ (168.1 g, 0.786 mol). This mixture was stirred at room temperature overnight. The reaction mixture was filtered to remove insoluble matter to afford filtrate A. The collected solids were washed with acetone (2×100 mL) and this filtrate was combined with filtrate A. The combined filtrates were concentrated under reduced pressure to 600 mL to afford a precipitate that was recovered by filtration. The collected solids were air-dried to give 110 g of crude material. This crude material was suspended in water (100 mL) and 5N NaOH was added until the pH was 12.5. The resulting suspension was filtered and the filtrate was treated with decolorizing carbon (Darco 6-60). The mixture was filtered and the filtrate was diluted with 10N H₂SO₄ until the pH was 5.0 to precipitate the intermediate title compound. This precipitate was collected by filtration and dried under reduced pressure to provide the intermediate title compound, 4-{2-[(methylsulfonyl)amino]ethyl}benzene boronic acid, (41.9 g, 82.5%) as a white powder.

¹H NMR (acetone-d₆, 300 MHz) δ 7.82 (d, 2H, J=8.4), 7.27 (d, 2H, J=7.8), 7.11 (s, 2H), 6.03 (m, 1H), 3.36 (m, 2H), 2.91 (m, 2H), 2.84 (s, 3H).

Preparation of Final Title Compound.

An aqueous solution of potassium formate was prepared in the following manner. To 15 mL of water was added KOH (85% flakes, 6.73 g, 0.102 mol), then 98% formic acid (4.70 g, 0.102 mol). Alternatively, one may use commercially available potassium formate. To this solution was then added K₂CO₃ (2.76 g, 0.0210 mol), 4-{2-[(methylsulfonyl)amino]ethyl}benzene boronic acid (4.62 g, 0.190 mol), 1-propanol (100 mL), and [(2R)-2-(4-iodophenyl)propyl][(methylethyl)sulfonyl]amine (7.35 g, 0.200 mol). This mixture was deoxygenated via three vacuum/N₂-refill cycles. Palladium black (0.0215 g, 0.0002 mol) was added and the mixture was again deoxygenated via three vacuum/N₂-refill cycles. The reaction flask was heated in a preheated oil bath at 88° C. and the mixture was stirred overnight.

HPLC analysis showed complete consumption of 4-{2-[(methylsulfonyl)amino]ethyl}benzene boronic acid, and the mixture was diluted with ethyl acetate and filtered through Celite® to remove palladium. The mixture was concentrated under reduced pressure and the resulting residue was partitioned between ethyl acetate and water. The organic phase was concentrated and the solid residue was collected and recrystallized from 1:1 acetone/water to afford the final title compound, {(2R)-2-[4-(4-{2-[(methylsulfonyl)amino]ethyl}phenyl)phenyl]propyl}[(methylethyl)sulfonyl]amine, (6.2 g, 75%) as a white crystalline powder.

¹H NMR (CDCl₃, 300 MHz) δ 7.54 (dd, 4H, J=1.8, 8.1), 7.29 (dd, 4H, J=1.8, 8.1), 4.27 (t, 1H, J=6.6), 3.91 (m, 1H), 3.43 (q, 2H, J=6.6), 3.37 (dd, 1H, J=5.7, 7.5), 3.26 (m, 1H), 3.07 (m, 2H), 2.93 (t, 2H, J=6.6), 2.87 (s, 3H), 1.34 (d, 3H, J=7.2), 1.31 (d, 3H, J=6.9), 1.27 (d, 3H, J=6.6).

Additional Preparation of {(2R)-2-[4-(4-{2-[(methylsulfonyl)amino]ethyl}phenyl)phenyl]propyl}[(methylethyl)sulfonyl]amine.

Within a single-neck, 3L round bottom flask equipped with a magnetic stir bar was placed potassium formate (112.8 g, 1.34 moles, 5.1 eq) and water (200 mL) to provide a pH 8 solution. Potassium carbonate (72.7 g, 0.526 mol, 2.0 eq), and 4-{2-[(methylsulfonyl)amino]ethyl}benzene boronic acid (60.8 g, 0.250 mol, 0.95 eq) was added to form a stirring suspension as 1-propanol (720 mL) was added. [(2R)-2-(4-iodophenyl)propyl][(methylethyl)sulfonyl]amine (96.6 g, 0.263 mol, 1.0 eq) was added followed by additional 1-propanol (600 mL). The resulting mixture was stirred for 3 minutes while the reaction flask was fitted with a heating mantle and a glycol-cooled reflux condenser. Vacuum (10-20 torr) was slowly applied to the system over 10 minutes. Stirring had stopped due to the additional precipitation of the cooled system; nevertheless, after 30 minutes, the system was returned to atmospheric pressure with nitrogen. With gentle heating, the flask was evacuated and refilled with nitrogen two additional times. Stirring was stopped and palladium black (0.28 g, 0.0026 mol, 0.01 eq) was quickly added to the flask. Stirring was resumed and the system was again evacuated and returned to atmospheric pressure with nitrogen over a 2 minute cycle. This evacuation/nitrogen purge was repeated two more times over a 15 second cycle and the mixture was heated to reflux.

After 16 hours, an aliquot was removed and analyzed by HPLC (275 nm detection). Analysis showed 0.07% of achiral dimer, (methylsulfonyl){2-[4-(4-{2-[(methylsulfonyl)amino]ethyl}phenyl)phenyl]ethyl}amine, relative to the desired product, {(2R)-2-[4-(4-{2-[(methylsulfonyl)amino]ethyl}phenyl)phenyl]propyl}[(methylethyl)sulfonyl]amine. The reaction mixture was cooled to 50° C. and ethyl acetate (500 mL) was added. The reaction mixture was then cooled to room temperature and the product, {(2R)-2-[4-(4-{2-[(methylsulfonyl)amino]ethyl}phenyl)phenyl]propyl}[(methylethyl)sulfonyl]amine, began to precipitate. Additional ethyl acetate (1 L) was introduced to redissolve the product and the upper organic phase was decanted and filtered through Celite® to remove palladium metal. The filter cake was rinsed with 1-propanol. The homogeneous filtrate was concentrated under reduced pressure to remove n-propanol and after removal of 1.5 L of distillate, the product suspension was filtered. The combined filter cakes were dried to afford 109.8 g of crude final title compound.

Recrystallization: The crude final title compound (109.8 g) was dissolved in acetone (490 mL). This solution was filtered though a glass filter to retain a minor amount of dark insoluble material. To the slowly stirred filtrate was added water (300 mL) over 15 min. The resulting suspension was stirred for 15 minutes and additional water (20 mL) was introduced over 10 minutes. The suspension was subsequently stirred for 30 minutes at room temperature and was filtered.

The cake was washed with 1:1 acetone/water (600 mL) and was dried at 35° C. overnight. This process afforded 80.3 g (81.1%) of {(2R)-2-[4-(4-{2-[(methylsulfonyl)amino]ethyl}phenyl)phenyl]propyl}[(methylethyl)sulfonyl]amine as a white crystalline powder with a mean particle size of about 29 to about 34 microns. HPLC analysis indicated 0.01% achiral dimer, (methylsulfonyl){2-[4-(4-{2-[(methylsulfonyl)amino]ethyl}phenyl)phenyl]ethyl}amine, and 0.02% chiral dimer, ((2R)-2-{4-[(4-((1R)-1-methyl-2-{[(methylethyl)sulfonyl]amino}ethyl)phenyl]phenyl}propyl)[(methylethyl)sulfonyl]amine.

### EXAMPLE 2

Alternative Preparation of {(2R)-2-[4-(4-{2-[(methylsulfonyl)amino]ethyl}phenyl)phenyl]propyl}[(methylethyl)sulfonyl]amine.

Preparation of 4-{2-[(tert-butoxy)-N-(methylsulfonyl)carbonylamino]ethyl}benzene boronic Acid.

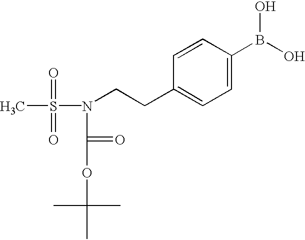

To a room temperature solution of (tert-butoxy)-N-(methylsulfonyl)-N-{2-[4-(4,4,5,5-tetramethyl(1,3,2-dioxaborolan-2-yl))phenyl]ethyl}carboxamide (81.0% potent, 95 g, 0.18 mol, prepared in example 1) in acetone (2 L) was added 1N ammonium acetate (1L) and sodium periodate (145 g, 0.678 mol) with stirring. The reaction was allowed to proceed overnight. The reaction mixture was concentrated to remove the acetone, and the aqueous phase was decanted away from the oily product. The aqueous phase was extracted with CH₂Cl₂ (100 mL) and MTBE (2×100 mL). The combined oily product and organic phases were adjusted to pH 12.5 with the addition of 1 N NaOH. The phases were separated, and the organic phase was extracted with 1 N NaOH (100 mL) and water (2×100 mL). HPLC analysis (60% CH₃CN/40% H₂O, 2 mL/min, Zorbax C-18, 205 nm) of the organic phase indicated that the product had been removed from this phase. The aqueous phases (containing product) were finally combined and washed with CH₂Cl₂ (100 mL) and MTBE (2×100 mL). The aqueous phase was added to CH₂Cl₂ (450 mL) and 1 N H₂SO₄ was added until the aqueous phase was at pH 3.05. The phases were separated and the aqueous phase was extracted with CH₂Cl₂ (100 mL). The combined organic extracts (containing product) were concentrated to an oil (58.5 g) that crystallized overnight. The resulting solid mass was triturated with 10% MTBE in heptane (100 mL) to afford, after filtration and drying under reduced pressure, the intermediate title compound, 4-{2-[(tert-butoxy)-N-(methylsulfonyl)carbonylamino]ethyl}benzene boronic acid, (47.7 g, 77.2%) as a white powder.

¹H NMR (d₆-DMSO, 300 MHz) δ 7.83 (d, 2H, J=4.8), 7.24 (d, 2H, J=5.1), 7.12 (s, 2H), 3.90 (t, 2H, J=3.9), 3.12 (s, 3H), 2.95 (t, 2H, J=4.5), 1.52 (s, 9H).

Preparation of Final Title Compound.

Run 1. Within a 3-neck, 1000 mL round-bottom flask was placed [(2R)-2-(4-iodophenyl)propyl][(methylethyl)sulfonyl]amine (15.0 g, 0.0408 mol, prepared in example 1), 4-{2-[(tert-butoxy)-N-(methylsulfonyl)carbonylamino]ethyl}benzene boronic acid (19.1 g, 0.0557 mol), K₂CO₃ (6.8 g, 0.0490 mol) and 1-propanol (300 mL). To this mixture was then added water (42 mL) and finally Pd(OAc)₂ (18 mg, 8.17×10⁻⁵ mol, 0.2 mol %). The resulting clear, pale amber solution was heated to reflux (87° C.) to become a dark amber, then a clear olive solution with stirring black particulates (Pd°). The reaction was allowed to stir for 20 h and was allowed to cool to room temperature. TLC analysis (1:9 EtOAc/CH₂Cl₂) of the resulting off-white suspension indicated desired product (R_{f} 032), complete consumption of [(2R)-2-(4-iodophenyl)propyl][(methylethyl)sulfonyl]amine (R_{f} 0.60) and only a trace of 4-{2-[(tert-butoxy)-N-(methylsulfonyl)carbonylamino]ethyl}benzene boronic acid (R_{f} 0.49). The suspension was diluted with EtOAc (300 mL) to give a clear, pale yellow solution that was filtered through Celite® (presaturated with EtOAc).

After washing the Celite® through with EtOAc, the filtrate was combined with that of an identical Run 2 which was conducted identically as described above. The combined filtrates from both runs were concentrated under reduced pressure to afford white solids that were diluted with EtOAc (1 L) and 10% K₂CO3 (300 mL) to form a clear, amber biphasic solution that was agitated. The aqueous phase (light pink) was separated and the organic phase was washed with additional 10% K₂CO₃ (4×300 mL). The aqueous phase was back extracted with EtOAc (300 mL) and the combined organic phases (1500 mL) were dried (MgSO₄), filtered, and concentrated to a volume of about 620 mL within a 3 L round-bottom flask. The clear, pale yellow solution was stirred slowly while heating to 60° C. Heptane (400 mL) was added dropwise from a separatory funnel to the stirring EtOAc solution at 60° C. (17 volumes of EtOAc/11 volumes of heptane). The heptanes were added over a period of 1.5 h and the clear, pale yellow solution was allowed to cool slowly with slow stirring overnight. The resulting white crystalline solids were cooled to 0° C., filtered, and washed with a minimum of 1:1 EtOAc/heptanes to afford the final title compound, {(2R)-2-[4-(4-{2-[(methylsulfonyl)amino]ethyl}phenyl)phenyl]propyl}[(methylethyl)sulfonyl]amine, (27.1 g, 75.7%) as a white crystalline powder.

Alternative Preparation of ((2R)-2-phenylpropyl)[(methylethyl)sulfonyl]amine.

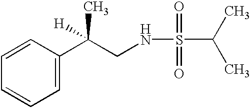

Preparation of (2R)-2-phenylpropan-1-ol.

An oven dried 500.0 mL three necked round bottom flask equipped with a mechanical stirrer, thermometer, addition funnel with a continuous nitrogen blanket is charged with 2.0 M solution of trimethylaluminum (65.6 mL, 131.2 mmol) and toluene (75.0 mL). Reaction solution was then chilled to −60° C. with dry ice/acetone bath. To this solution was then added R-styrene oxide dissolved in 100.0 mL of toluene over a period of 50.0 minutes (reaction is quite exothermic and can be controlled by the rate of addition of substrate). After stirring at this temperature for 60.0 minutes, reaction was brought to room temperature and stirred for 4.0 hours. Reverse quenched reaction at room temperature into a slurry of THF (100.0 mL) and sodium sulfate decahydrate (46.0 g) very cautiously over a period of 90.0 minutes (quenching was quite exothermic with evolution of gas). Filtered the precipitate formed over hyflo, then concentrated filtrate to provide the intermediate title compound, (2R)-2-phenylpropan-1-ol, (11.03 g, 92.6%) as an oil; 1H nmr (CDCl₃) δ 1.28-1.29 (d, 3H, J=6.9 Hz), 1.5 (b, 1H), 2.9-3.0 (m, 1H), 3.69-3.70 (d, 2H, J=6.64 Hz), 7.24-7.35 (aromatic); 13C nmr (CDCl₃) δ 18.31, 43.15, 69.40, 127.38, 128.20, 129.26144.39.

Preparation of 2-((2R)-2-phenylpropyl)isoindoline-1,3-dione

An oven dried 250.0 mL three necked round bottom flask equipped with a mechanical stirrer, thermometer, addition funnel with a continuous nitrogen blanket is charged with (2R)-2-phenylpropan-1-ol (2.0 mL, 14.32 mmol), phthalimide (2.1 g, 14.32 mmol), triphenylphosphine (5.63 g, 21.48 mmol) and THF (70.0 mL). To this solution at room temperature was then added a solution of diethylazodicarboxylate (3.38 mL, 21.48 mmol) dissolved in THF (10.0 mL) over a period of 15-20 minutes (reaction exothermed slightly to 50° C. by the end of addition went from clear to reddish color). Stirred reaction to room temperature overnight). To the red solution was added water (50.0 mL) and the organic extracted with chloroform (140.0 mL). Dried the organic solution with anhydrous magnesium sulfate, filtered and concentrated under reduced pressure to an oil. To the oil was added heptane (150.0 mL) with stirring. Filtered of precipitates, then concentrated filtrate to an oil. Plug filtration of the oil over silica gel with 1:1 ethylacetate/hexane and concentrating product fractions afforded the intermediate title compound, 2-((2R)-2-phenylpropyl)isoindoline-1,3-dione, (4.27 g, 96%) as an oil which solidified on equilibrating to room temperature; 1H nmr (CDCl₃) δ 1.3 (d, 3H), 3.3-4.0(m,1H), 3.7-3.9 (m, 2H), 7.1-7.3 (aromat. m, 2H), 7.63-7.7 (aromat. m, 2H), 7.8-7.85 (aromat. m, 4H).

Preparation of (2R)-2-phenylpropylamine.

A 500 mL three necked round bottom flask equipped with a mechanical stirrer, thermometer and addition funnel is charged with 2-((2R)-2-phenylpropyl)isoindoline-1,3-dione (11.54 g, 43.49 mmol), toluene (200.0 mL) and anhydrous hydrazine (2.73 mL, 86.99 mmol). Reaction is then stirred at room temperature for 3.0 hours and then heated at 90° C.-95° C. for 2.0 hours. Cooled the slurry to room temperature, filtered precipitates, then concentrated filtrate to provide the intermediate title compound, (2R)-2-phenylpropylamine, (5.58 g, 94.9%) an oil; 1H nmr (CDCl₃) δ 1.21 (d, 3H), 1.40-1.60 (b, 2H), 2.68-2.80 (m, 1H), 2.81-2.87 (m, 2H) 7.20 (m, 2H), 7.32 (m, 2H).

Preparation of Final Title Compound.

To a solution of the (2R)-2-phenylpropylamine (1.2 g, 8.87 mmol) in hexane (16.0 mL) was added triethylamine (2.47 mL, 17.74 mmol) and dimethylaminopyridine (0.30 g, 2.47 mmol). Cooled reaction to 5° C., then added a solution of isopropylsulfonyl chloride (0.97 mL, 8.69 mmol) dissolved in methylene chloride (6.0 mL) over a period of 15.0 minutes. Stirred for 45.0 minutes, then stirred at room temperature for 120.0 minutes. Quenched reaction with 1N HCl (20.0 mL) and extracted organic with methylene chloride (25.0 mL). Dried organic layer with anhydrous magnesium sulfate, filtered and concentrated filtrate to provide the final title compound, ((2R)-2-phenylpropyl)[(methylethyl)sulfonyl]amine, (1.93 g, 90.1%) an oil; 1H nmr (CDCl₃) δ 1.25 (d, 3H, J=6.9 Hz), 1.29(d, 3H, J=6.9 Hz), 1.30 (d, 3H, J=7.2 Hz), 2.98 (m, 1H), 3.05 (m, 1H), 3.22 (m, 1H), 3.36 (m, 1H), 3.89 (b, 1H), 7.23 (m, 2H), 7.34 (m, 2H).

### EXAMPLE 2

Preparation of N-2-(4-(3-thienyl)phenylpropyl 2-propanesulfonamide (392098).

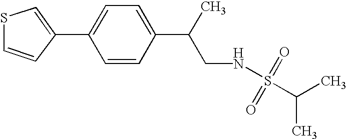

The title compound is prepared in a manner analogous to the procedure set forth in WO 98/33496 published Aug. 6, 1998, Example 28.

### EXAMPLE 3

Preparation of [2-fluoro-2-(4-{3-[(methylsulfonyl)amino]phenyl}phenyl)propyl][(methylethyl)sulfonyl]amine.

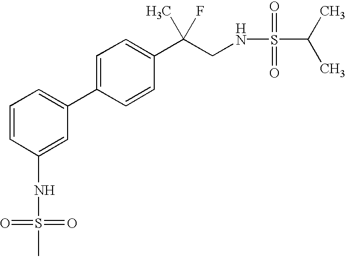

Preparation of 1-amino-2-(4-iodophenyl)propan-2-ol.

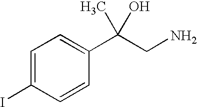

The trimethylsilyl-protected cyanohydrin derivative of 4-iodoacetophenone was prepared in situ following generally the method disclosed by Greenlee and Hangauer, Tetrahedron Lett., 24(42), 4559 (1983). Accordingly, cyanotrimethylsilane (21.4 g, 0.216 mol) was added dropwise over 5 minutes to a dry, room temperature solution containing 4-iodoacetophenone (44.3 g, 0.180 mol), 18-crown-6 (1.6 g, 6.1 mmoles) and KCN (1.17 g, 0.018 mol) in THF (100 mL). The resulting solution was allowed to stir for 2.5 h. TLC analysis (3:7 EtOAc/Hexanes) showed consumption of starting acetophenone.

A 10M solution of borane in dimethylsulfide (25 mL, 0.25 mol) was added rapidly to the reaction solution and the resulting mixture was heated at reflux for 16 h. The mixture was cooled to room temperature and anhydrous 10% (by wt) HCl in methanol was added slowly over 1 h (GAS EVOLUTION). The solution was allowed to stir for an additional hour, and was concentrated under reduced pressure to give the crude title compound as white solid and as the hydrochloride salt. This salt was triturated with methyl t-butyl ether and filtered. The free base was prepared by adding 1 N NaOH to a suspension of the HCl salt in CH₂Cl₂ (150 mL) and THF (350 mL) until pH 12.3 was reached. The phases were separated and the organic phase was washed with brine (25 mL). The organic phase containing the free amine was concentrated under reduced pressure and the resulting solids were triturated with diethyl ether (30 mL) to afford 1-amino-2-(4-iodophenyl)propan-2-ol (35.6 g, 71.3%) as an off-white powder after vacuum drying. ¹H NMR (CD₃OD, 300 MHz): δ 7.68 (d, 2H, J=8.4), 7.24 (d, 2H, J=8.7), 2.78 (m, 2H), 1.46 (s, 3H).

Preparation of [2-hydroxy-2-(4-iodophenyl)propyl][(methylethyl)sulfonyl]amine.

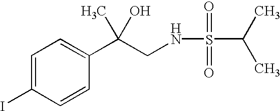

Into a 250 mL 3 necked flask fitted with a stirrer and thermometer, was added dropwise 2-propanesulfonyl chloride (1.60 g, 0.011 mol) to 1-amino-2-(4-iodophenyl)propan-2-ol (2.77 gm, 0.01 mol) in 125 mL CH₂Cl₂ while stirring at 0° C. under nitrogen. The reaction was then allowed to warm to room temperature and stirred overnight at this temperature. In the morning, the mixture was poured into H₂O and the layers were separated. The organic layer was washed once with H₂O, dried over anhydrous Na₂SO₄, filtered, and concentrated under reduced vacuum. The resulting semi-solid was purified via silica gel chromatography employing the Prep. LC-2000 and eluting with a solvent of Hexane/EtOAc 3:1 to provide [2-hydroxy-2-(4-iodophenyl)propyl][(methylethyl)sulfonyl]amine (744 mg, 19%) as a solid material. FDMS 382 (M*).

Analysis for C₁₂H₁₈NO₃ S I:

Theory: C, 37.61 H, 4.73 N, 3.65

Found: C, 38.08 H, 4.26 N, 3.55

Alternative Preparation of [2-hydroxy-2-(4-iodophenyl)propyl][(methylethyl)sulfonyl]amine.

In a 250 mL-3 neck flask fitted with a stirrer and thermometer, 2.10 g. of propanesulfonyl chloride was added dropwise to 2.77 g. of 1-amino-2-(4-iodophenyl)propan-2-ol and 2.30 g. of DBU in CH₂Cl₂ (150 mL) while stirring at 0° C. under a nitrogen atmosphere. The reaction was allowed to warm to room temperature and stirred overnight at this temperature. In the morning, the reaction was diluted with CH₂Cl₂ (100 mL) and the organic layer was washed two times with H₂O, dried over anhydrous Na₂SO₄, filtered, and concentrated under reduced vacuum to yield a viscous oil. This material was purified via silica gel chromatography employing the Chromatotron, using a 4000 micron rotor and eluting with a solvent of methylene chloride/methanol 19:1 to yield [2-hydroxy-2-(4-iodophenyl)propyl][(methylethyl)sulfonyl]amine (1.0 g, 31%) as a viscous oil. Ion spray M.S. 382 (M*−1).

Preparation of [2-fluoro-2-(4-iodophenyl)propyl[](methylethyl)sulfonyl]amine.

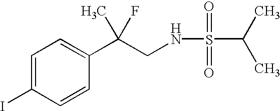

Into a 10 mL single neck flask, a solution of [2-hydroxy-2-(4-iodophenyl)propyl][(methylethyl)sulfonyl]amine (158 mg, 0.41 mmol) in 1.7 mL CH₂Cl₂ was added syringe wise slowly to a solution of DAST (66 mg, 0.41 mmol) in 0.3 mL CH₂Cl₂ while stirring at −78° C. under nitrogen. The reaction was then allowed to warm to room temperature and the mixture was diluted with H₂O and CH₂Cl₂. The layers were separated and the organic layer was washed twice with H₂O, dried over anhydrous Na₂SO₄, filtered, and concentrated under reduced vacuum to [2-fluoro-2-(4-iodophenyl)propyl][(methylethyl)sulfonyl]amine (113 mg) as a solid. Ion spray M.S. 384 (M*−1).

Additional Preparation of [2-fluoro-2-(4-iodophenyl)propyl][(methylethyl)sulfonyl]amine.

Into a 100 mL 3-neck flask fitted with a stirrer and thermometer, 1.0 g. of [2-hydroxy-2-(4-iodophenyl)propyl][(methylethyl)sulfonyl]amine in CH₂Cl₂ (15 mL) was added dropwise to 0.3 mL DAST in CH₂CL₂ (10 mL) while stirring at −78° C. under a nitrogen atmosphere. Reaction was allowed to warm to room temperature and diluted with CH₂Cl₂ (50 mL). This organic layer was washed with H₂O, dried over anhydrous Na₂SO₄, filtered, and concentrated under reduced vacuum to yield an oil. This material was purified via silica gel chromatography employing the Chromatotron and using a 4000 micron rotor while eluting with a gradient solvent of hexane/ethyl acetate 9:1 to hexane/ethyl acetate 3:1 to yield [2-fluoro-2-(4-iodophenyl)propyl][(methylethyl)sulfonyl]amine (0.906 g) as a white solid. Ion spray M.S. 384 (M*−1).

Analysis for C₁₂H₁₇NO₂SFI:

Theory: C, 37.42 H, 4.44 N, 3.64

Found: C, 37.27 H, 4.33 N, 3.61

Preparation of {2-[4-(3-aminophenyl)phenyl]-2-fluoropropyl}[(methylethyl)sulfonyl]amine.

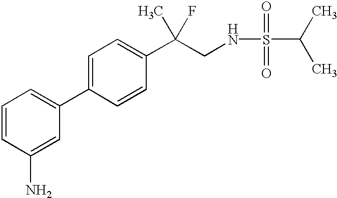

Into a 50 mL single neck flask [2-fluoro-2-(4-iodophenyl)propyl][(methylethyl)sulfonyl]amine (200 mg, 0.53 mmol), 3-aminobenzene boronic acid (188 mg, 0.76 mmol), potassium carbonate (104 mg, 0.76 mmol) and tetrakis(triphenyl phosphine)palladium(0) (41 mg, 0.036 mmol) were combined in dioxane/water (20 mL, 3:1). The mixture was heated at 100° C. under stirring for 18 hours. The reaction was cooled to room temperature and poured into H₂O. The desired product was extracted with ethyl acetate and the organic layer was separated and washed twice with H₂O, dried over K₂CO₃, and concentrated under reduced vacuum to yield the crude material (276 mg) as a dark oil. The resulting oil was purified via silica gel chromatography employing the Chromatotron using a 4000 micron rotor and eluting with a solvent of Hexane/Ethyl Acetate 1:1 to yield the title compound (164 mg, 90%) as a viscous oil. Ion spray M.S. 351.4 (M*+1).

Analysis calculated for: C₁₈H₂₃N₂ O₂ S F:

Theory: C, 61.69 H, 6.62 N, 7.99

Found: C, 61.53 H, 6.55 N, 8.13

Preparation of Final Title Compound.

A 50 mL flask fitted with a stirrer and thermometer was charged with DBU (67 mg, 1.1 eq), {2-[4-(3-aminophenyl)phenyl]-2-fluoropropyl}[(methylethyl)sulfonyl]amine (140 mg, 0.44 mmol) and methylene chloride (10 mL) under an atmosphere of nitrogen, and cooled to 0° C. To this stirring solution was added dropwise chloro-methane sulfonyl chloride (69 mg, 1.5 eq). The reaction was allowed to warm to room temperature and stirred overnight at this temperature. In the morning, the mixture was poured into H₂O and the layers were separated. The organic layer was washed once with H₂O, dried over anhydrous Na₂SO₄, filtered, and concentrated under reduced vacuum to yield the crude material (192 mg) as a yellow oil. This crude material was purified via silica gel chromatography employing the Chromatotron using a 4000 micron rotor and eluting with a solvent of Methylene Chloride/ethyl acetate 9:1 to yield the final title compound, [2-fluoro-2-(4-{3-[(methylsulfonyl)amino]phenyl}phenyl)propyl][(methylethyl)sulfonyl]amine, (50 mg, 29%) as a white foam. Ion spray mass spectra 427.1 (M*−1).

Analysis for C₁₉H₂₅N₂ O₄ S₂ F:

Theory: C, 53.25 H, 5.88 N, 6.54

Found: C, 53.56 H, 6.11 N, 6.29

### EXAMPLE 3a

Preparation of [2-Fluoro-2-(4-{3-[(methylsulfonyl)amino]phenyl}phenyl)propyl][(methylethyl)sulfonyl]amine (enantiomer 1).

Preparation of (+)-[2-fluoro-2-(4-iodophenyl)propyl][(methylethyl)sulfonyl]amine and (−)-[2-fluoro-2-(4-iodophenyl)propyl][(methylethyl)sulfonyl]amine.

[2-fluoro-2-(4-iodophenyl)propyl][(methylethyl)sulfonyl]amine (2.0 g, prepared in example 3) was dissolved into 3A ethanol (30 mL) and was further diluted with heptane (20 mL). [As used herein the term “3A ethanol” refers to ethanol containing 5% methanol.] The mixture was agitated via ultrasound to form a clear, colorless solution. This lot was loaded upon a 8×28 cm preparative Chiralpak AD chromatographic column that was pre-equilibrated with 60% 3A ethanol/40% heptane. Eluent flow was 300 mL/min and detection wavelength was 240 nm. The first eluting substance was (+)-[2-fluoro-2-(4-iodophenyl)propyl][(methylethyl)sulfonyl]amine, [α]_{D}=+18.5 (c=1.08, MeOH), and the subsequent eluting substance was (−)-[2-fluoro-2-(4-iodophenyl)propyl][(methylethyl)sulfonyl]amine, [α]_{D}=−23.5 (c=1.02, MeOH). The above procedure was repeated twice in an analogous manner with [2-fluoro-2-(4-iodophenyl)propyl][(methylethyl)sulfonyl]amine (second run, 3.0 g dissolved in 50 mL 3A ethanol/heptane, 3:2 and a third run, 2.0 g dissolved in 0.8 g dissolved in 40 mL 3A ethanol/heptane, 3:2). Thus, in three runs, a total of 5.8 g of [2-fluoro-2-(4-iodophenyl)propyl][(methylethyl)sulfonyl]amine was resolved into its component enantiomers in the following yields after concentration (in vacuo) of fractions:

(+)-[2-fluoro-2-(4-iodophenyl)propyl][(methylethyl)sulfonyl]amine (2.38 g, 41.0%);

(−)-[2-fluoro-2-(4-iodophenyl)propyl][(methylethyl)sulfonyl]amine (1.2 g, 20.7%).

Analysis conditions: 0.46×35 cm Chiralpak AD 60% ethanol (5% methanol)/40% Heptane; Flow: 1.0 mL/min, detection wavelength: 240 nm.

For (+)-[2-fluoro-2-(4-iodophenyl)propyl][(methylethyl)sulfonyl]amine: Rₜ=5.4 min, MS (ES+) 384 (M−1).

¹H NMR (CDCl₃, 300 MHz): δ 7.73 (d, 2H, J=8.1), 7.09 (d, 2H, J=8.4), 4.27 (t, 1H, J=6.2), 3.50 (m, 2H), 3.03 (m, 1H), 1.69 (d, 3H, J=22), 1.30 (d, 3H, J=7), 1.27 (d, 3H, J=7).

Analysis for C₁₂H₁₇FINO₂S:

Theory: C 37.41, H 4.45, N 3.64.

Found: C 37.54, H 4.43, N 3.64.

For (−)-[2-fluoro-2-(4-iodophenyl)propyl][(methylethyl)sulfonyl]amine: Rₜ=10.1 min. MS (ES+) 384 (M−1).

¹H NMR spectrum identical to that of (+)-[2-fluoro-2-(4-iodophenyl)propyl][(methylethyl)sulfonyl]amine.

Analysis for C₁₂H₁₇FINO₂S:

Theory: C 37.41, H 4.45, N 3.64.

Found: C 37.56, H 4.43, N 3.59.

(+)-[2-Fluoro-2-(4-iodophenyl)propyl][(methylethyl)sulfonyl]amine (300 mg, 0.78 mmol), the borate of formula:

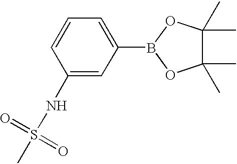

(347 mg, 1.5 eq.), potassium carbonate (156 mg, 1.5 eq), tetrakis(triphenyl phosphine)palladium(0) (75 mg, 0.06 mmol) and dioxane/water (36 mL, 3:1) were mixed together in a 100 mL single neck flask and stirred at 80° C. for 4 hours. The reaction was cooled to room temperature and poured into H₂O and the desired product was extracted with ethyl acetate. The organic layer was backwashed once with H₂O, dried over K₂CO₃, filtered, and concentrated under reduced pressure to yield 191 mg as a viscous oil. This material was purified via silica gel chromatography employing the chromatotron and using a 2000 micron rotor while eluting with a solvent of hexane/ethyl acetate 1:1 to yield the title compound (86 mg, 26%) as a white solid. Ion spray M.S. 427.1 (M*−1).

Calculated for: C₁₉H₂₅N₂O₄S₂ F—H₂O:

Theory: C 51.08, H 6.09, N 6.27.

Found: C 51.29, H 5.63, N 6.29.

### EXAMPLE 3b

Preparation of [2-Fluoro-2-(4-{3-[(methylsulfonyl)amino]phenyl}phenyl)propyl][(methylethyl)sulfonyl]amine (enantiomer 2).

(−)-[2-Fluoro-2-(4-iodophenyl)propyl][(methylethyl)sulfonyl]amine (493 mg, 1.28 mmol, prepared in example 3a), the borate of formula:

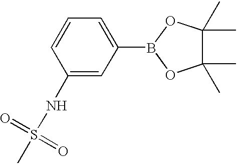

(385 mg, 1.30 mmol), 2.0 M Na₂CO₃/H₂O (2.2 mL, excess), tetrakis(triphenyl phosphine)palladium(0) (100 mg, 0.09 mmol) and dioxane (15 mL) were mixed together in a 50 mL single neck flask and stirred at 80° C. overnight. In the morning the reaction was cooled to room temperature and poured into H₂O and the desired product was extracted with ethyl acetate. The organic layer was backwashed once with H₂O, dried over K₂CO₃, filtered, and concentrated under reduced pressure to yield 571 mg as a foam. This material was purified via silica gel chromatography employing the chromatotron and using a 4000 micron rotor while eluting with a solvent of hexanelethyl acetate 1:1 to yield the title compound (294 mg, 56%) as a brown solid. Ion spray M.S. 427.3 (M*−1).

Calculated for: C₁₉H₂₅N₂O₄S₂ F—H₂O:

Theory: C 51.08, H 6.09, N 6.27.

Found: C 51.29, H 5.63, N 6.29.

The ability of compounds to potentiate glutamate receptor-mediated response may be determined using fluorescent calcium indicator dyes (Molecular Probes, Eugene, Oreg., Fluo-3) and by measuring glutamate-evoked efflux of calcium into GluR4 transfected HEK293 cells, as described in more detail below.

In one test, 96 well plates containing confluent monolayers of HEK 293 cells stably expressing human GluR4B (obtained as described in European Patent Application Publication Number EP-A1-583917) are prepared. The tissue culture medium in the wells is then discarded, and the wells are each washed once with 200 μl of buffer (glucose, 10 mM, sodium chloride, 138 mM, magnesium chloride, 1 mM, potassium chloride, 5 mM, calcium chloride, 5 mM, N-[2-hydroxyethyl]-piperazine-N-[2-ethanesulfonic acid], 10 mM, to pH 7.1 to 7.3). The plates are then incubated for 60 minutes in the dark with 20 μM Fluo3-AM dye (obtained from Molecular Probes Inc., Eugene, Oreg.) in buffer in each well. After the incubation, each well is washed once with 100 μl buffer, 200 μl of buffer is added and the plates are incubated for 30 minutes.

Solutions for use in the test are also prepared as follows. 30 μM, 10 μM, 3 μM and 1 μM dilutions of test compound are prepared using buffer from a 10 mM solution of test compound in DMSO. 100 μM cyclothiazide solution is prepared by adding 3 μl of 100 mM cyclothiazide to 3 ml of buffer. Control buffer solution is prepared by adding 1.5 μl DMSO to 498.5 μl of buffer.

Each test is then performed as follows. 200 μl of control buffer in each well is discarded and replaced with 45 μl of control buffer solution. A baseline fluorescent measurement is taken using a FLUOROSKAN II fluorimeter (Obtained from Labsystems, Needham Heights, Mass., USA, a Division of Life Sciences International Plc). The buffer is then removed and replaced with 45 μl, of buffer and 45 μl of test compound in buffer in appropriate wells. A second fluorescent reading is taken after 5 minutes incubation. 15 μl of 400 μM glutamate solution is then added to each well (final glutamate concentration 100 μM), and a third reading is taken. The activities of test compounds and cyclothiazide solutions are determined by subtracting the second from the third reading (fluorescence due to addition of glutamate in the presence or absence of test compound or cyclothiazide) and are expressed relative to enhance fluorescence produced by 100 μM cyclothiazide.

In another test, HEK293 cells stably expressing human GluR4 (obtained as described in European Patent Application Publication No. EP-A1-0583917) are used in the electrophysiological characterization of AMPA receptor potentiators. The extracellular recording solution contains (in mM): 140 NaCl, 5 KCl, 10 HEPES, 1 MgCl2, 2 CaCl2, 10 glucose, pH=7.4 with NaOH, 295 mOsm kg-1. The intracellular recording solution contains (in mM): 140 CsCl, 1 MgCl2, 10 HEPES, (N-[2-hydroxyethyl]piperazine-N1-[2-ethanesulfonic acid]) 10 EGTA (ethylene-bis(oxyethylene-nitrilo)tetraacetic acid), pH=7.2 with CsOH, 295 mOsm kg-1. With these solutions, recording pipettes have a resistance of 2-3 MΩ. Using the whole-cell voltage clamp technique (Hamill et al.(1981)Pflügers Arch., 391: 85-100), cells are voltage-clamped at −60 mV and control current responses to 1 mM glutamate are evoked. Responses to 1 mM glutamate are then determined in the presence of test compound. Compounds are deemed active in this test if, at a test concentration of 10 μM or less, they produce a greater than 10% increase in the value of the current evoked by 1 mM glutamate and this effect can be blocked by a specific AMPA receptor antagonist such as NBQX.

In order to determine the potency of test compounds, the concentration of the test compound, both in the bathing solution and co-applied with glutamate, is increased in half log units until the maximum effect was seen. Data collected in this manner are fit to the Hill equation, yielding an EC50 value, indicative of the potency of the test compound. Reversibility of test compound activity is determined by assessing control glutamate 1 mM responses. Once the control responses to the glutamate challenge are re-established, the potentiation of these responses by 100 μM cyclothiazide is determined by its inclusion in both the bathing solution and the glutamate-containing solution. In this manner, the efficacy of the test compound relative to that of cyclothiazide can be determined.

The diagnosis of depression is made primarily by quantification of alterations in patients' mood. These evaluations of mood are generally performed by a physician or quantified by a neuropsychologist using validated rating scales, such as the Hamilton Depression Rating Scale or the Brief Psychiatric Rating Scale which are well known to one of ordinary skill in the art. Numerous other scales have been developed to quantify and measure the degree of mood alterations in patients with depression, such as insomnia, difficulty with concentration, lack of energy, feelings of worthlessness, and guilt.

The standards for diagnosis of depression as well as all psychiatric diagnoses are collected in the Diagnostic and Statistical Manual of Mental Disorders (Fourth Edition) referred to as the DSM-IV manual published by the American Psychiatric Association, 1994.

Certain behavioral despair animal models are predictive of antidepressant activity in man, such as the Forced Swim Test and the Tail Suspension Test. For example, see “Experimental Approaches to Anxiety and Depression”, Edited by J. M. Elliott, et al., (1992), John Wiley & Sons Ltd., Chapter 5, Behavioural Models of Depression, Porsolt and Lenegre, pages 73-85. The Forced Swim Test and the Tail Suspension Test are described in detail below.

Forced Swim Test (FST): A male mouse (for example, of the N1H-Swiss strain supplied by Harlan Sprague-Dawley) typically weighing 25-30 g is placed in a clear plastic cylinder (diameter: 10 cm; height: 25 cm) filled with 6 cm of water (22-25° C.) for six min. The duration of immobility during the last four minutes of the six minute test period is scored. A mouse is recorded as immobile when floating motionless or making only those movements necessary to keep its head above water. Administration of clinically effective antidepressants before this test (e.g. imipramine, 15 mg/kg, administered intraperitoneally 15 min prior to testing) typically produce a diminution in the time immobile (e.g. Trullas and Skolnick, European Journal of Pharmacology, 185, 1-10 (1990)).

To determine if augmentation of action is produced with a combination of a suitable AMPA receptor potentiator and a suitable antidepressant, as defined herein, mice can be injected intraperitoneally (in, e.g., 0.1 ml) with a suitable AMPA receptor potentiator and a suitable antidepressant (in e.g., 0.1 ml) at doses that do not individually produce a significant reduction in immobility. A combination of these compounds, if an augmentation (synergism) exists, would result in an effect greater than each agent alone. In variations of this procedure, increasing doses of either compound would be injected in the presence of a fixed (subeffective or marginally effective) dose of the second compound. An augmentation of action would be reflected by a reduction in immobility that is greater than the arithimetic sum of the effect of each agent alone. A hypothetical example follows: the vehicle injected animals have a mean immobility time of 130 seconds and animals injected with a standard dose of a clinically effective antidepressant have an immobility of 90 seconds. Injection of a suitable AMPA receptor potentiator at a dose that by itself does not significantly alter immobility time (e.g. 128 seconds) reduces immobility time to 65 seconds when combined with the standard dose of a clinically effective antidepressant. That is, the predicted arithmetic change would be (130−90)+(130−128)=42 seconds, but the combination of the agents yield a reduction of 130−65=65 seconds. Standard statistical tests can be used to determine if this difference is significant. The same general strategy can be applied to the forced swim test as carried out in rats (below) as well as the tail suspension test, also described below.

In a variant of the forced swim test procedure using rats, a rat (for example, a male Sprague Dawley rat from Harlan Sprague-Dawley) weighing 200-300 g is placed in a clear plastic cylinder (diameter: 18 cm; height: 40 cm) filled with water (22-25° C.) to a depth of 16 cm for fifteen min. After testing, the rat is dried with paper towels and placed in holding cages. Five minutes later, animals receive intraperitoneal injections (0.1 ml) of drugs or vehicle, and are then returned to their home cages. On the following day, rats receive a second dose of compound(s) or vehicle 1 h prior to the test. The rats are placed in cylinders as described above for 5 min and the duration of immobility recorded.

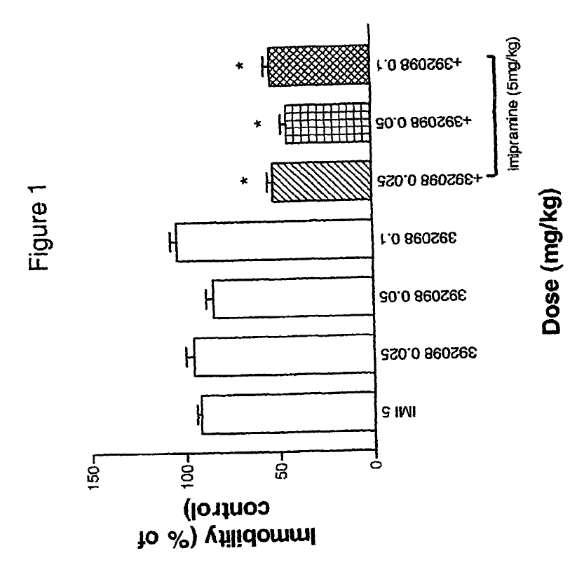

FIG. 1 discloses that low doses of 392098 by itself (0.025-0.1 mg/kg) produced no effect in the Forced Swim Test in mice. The minimum effective dose (MED) of 392098 was 0.5 mg/kg, i.p. However, FIG. 1 further reveals that when 392098 was combined at a dose as low as 25 micrograms/kg with a subeffective dose of imipramine (5 mg/kg), a statistically significant reduction in immobility of the mouse resulted. Moreover, FIG. 1 reveals an unexpected shift in the dose response curve of 392098 of at least 20-fold when it was administered in combination with a subeffective dose of imipramine.

Admininstration

The dosages of the drugs used in the present invention must, in the final analysis, be set by the physician in charge of the case, using knowledge of the drugs, the properties of the drugs in combination as determined in clinical trials, and the characteristics of the patient, including diseases other than that for which the physician is treating the patient. As used herein the term “effective amount” is the amount or dose of each component, the suitable antidepressant and the suitable AMPA receptor potentiator, which provides the desired effect in the patient under diagnosis or particular treatment, such as treatment for depression.

An effective amount can be readily determined by the attending diagnostician, as one skilled in the art, by the use of known techniques and by observing results obtained under analogous circumstances. In determining the effective amount or dose, a number of factors are considered by the attending diagnostician, including, but not limited to: the species of mammal; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

General outlines of the dosages, and some preferred dosages are set forth below.

A typical daily dose of the first component, which is a suitable antidepressant will contain from about 0.01 mg/kg to about 100 mg/kg of the first component. Preferably, daily doses will be about 0.05 mg/kg to about 50 mg/kg, more preferably from about 0.1 mg/kg to about 25 mg/kg. More specific dosages of certain suitable antidepressants are as follows:

Fluoxetine: from about 1 to about 80 mg, once/day; preferred, from about 10 to about 40 mg once/day;

Duloxetine: from about 1 to about 30 mg once/day; preferred, from about 5 to about 20 mg once/day;

Venlafaxine: from about 10 to about 150 mg once-thrice/day; preferred, from about 25 to about 125 mg thrice/day;

Milnacipran: from about 10 to about 100 mg once-twice/day; preferred, from about 25 to about 50 mg twice/day;

Citalopram: from about 5 to about 50 mg once/day; preferred, from about 10 to about 30 mg once/day;

Fluvoxamine: from about 20 to about 500 mg once/day; preferred, from about 50 to about 300 mg once/day;

Paroxetine: from about 20 to about 50 mg once/day; preferred, from about 20 to about 30 mg once/day.

Sertraline: from about 20 to about 500 mg once/day; preferred, from about 50 to about 200 mg once/day.

Reboxetine: from about 1 to about 30 mg, once to four times/day; preferred, from about 5 to about 30 mg once/day.

Buproprion: from about 100 to about 300 mg/day.

A typical daily dose of the second component which is a suitable AMPA receptor potentiator, will contain from about 5 micrograms to about 150 mg of the suitable AMPA receptor potentiator, preferably about 5 micrograms to about 50 mg of the suitable AMPA receptor potentiator.

In more general terms, one would create a combination of the present invention by choosing a dosage of first and second components according to the spirit of the above guideline.

As used herein the term “patient” refers to a mammal such as a dog, rat, mouse, human, and the like. The preferred patient is a human.

The adjunctive therapy of the present invention is carried out by administering the first component together with the second component in any manner which provides effective levels of the compounds in the body at the same time. The compounds concerned are normally administered orally, and so oral administration of the adjunctive combination is preferred. They may be administered together, in a single dosage form, or may be administered separately.

However, oral administration is not the only route or even the only preferred route. For example, transdermal administration may be very desirable for patients who are forgetful or petulant about taking oral medicine. One of the drugs may be administered by one route, such as oral, and the others may be administered by the transdermal, percutaneous, intravenous, intramuscular, intranasal or intrarectal route, in particular circumstances. The route of administration may be varied in any way, limited by the physical properties of the drugs and the convenience of the patient and the caregiver.

The adjunctive combination may be administered as a single pharmaceutical composition, and so pharmaceutical compositions incorporating both compounds are important embodiments of the present invention. Such compositions may take any physical form which is pharmaceutically acceptable, but orally usable pharmaceutical compositions are particularly preferred. Such adjunctive pharmaceutical compositions contain an effective amount of each of the compounds, which effective amount is related to the daily dose of the compounds to be administered. Each adjunctive dosage unit may contain the daily doses of all compounds, or may contain a fraction of the daily doses, such as one-third of the doses. Alternatively, each dosage unit may contain the entire dose of one of the compounds, and a fraction of the dose of the other compounds. In such case, the patient would daily take one of the combination dosage units, and one or more units containing only the other compounds. The amounts of each drug to be contained in each dosage unit depends on the identity of the drugs chosen for the therapy, and other factors such as the indication for which the adjunctive therapy is being given.

The inert ingredients and manner of formulation of the adjunctive pharmaceutical compositions are conventional, except for the presence of the combination of the present invention. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the type of composition to be used. The amount of the compounds, however, is best defined as the effective amount, that is, the amount of each compound which provides the desired dose to the patient in need of such treatment. The activity of the adjunctive combinations do not depend on the nature of the composition, so the compositions are chosen and formulated solely for convenience and economy. Any of the combinations may be formulated in any desired form of composition. Some discussion of different compositions will be provided, followed by some typical formulations.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant is necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acid contents of the stomach. Such formulations are created by coating a solid dosage form with a film of a polymer which is insoluble in acid environments, and soluble in basic environments. Exemplary films are cellulose acetate phthalate, polyvinyl acetate phthalate, hydroxypropyl methylcellulose phthalate and hydroxypropyl methylcellulose acetate succinate. It is preferred to formulate duloxetine and duloxetine-containing combinations as enteric compositions, and even more preferred to formulate them as enteric pellets.

A preferred duloxetine enteric formulation is a pellet formulation comprising a) a core consisting of duloxetine and a pharmaceutically acceptable excipient; b) an optional separating layer; c) an enteric layer comprising hydroxypropylmethylcellulose acetate succinate (HPMCAS) and a pharmaceutically acceptable excipient; d) an optional finishing layer. This enteric formulation is described in U.S. Pat. No. 5,508,276, herein incorporated by reference in its entirety.

Tablets are often coated with sugar as a flavor and sealant. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa bufter is a traditional suppository base, which may be modified by addition of waxes to raise its melting point slightly. Water-miscible suppository bases comprising, particularly, polyethylene glycols of various molecular weights are in wide use, also.

Transdermal patches have become popular recently. Typically they comprise a resinous composition in which the drugs will dissolve, or partially dissolve, which is held in contact with the skin by a film which protects the composition. Many patents have appeared in the field recently. Other, more complicated patch compositions are also in use, particularly those having a membrane pierced with innumerable pores through which the drugs are pumped by osmotic action.

What is claimed is:

1. A method for treating depression, comprising administering to a patient an effective amount of a first component which is a suitable antidepressant, in combination with an effective amount of a second component which is a suitable AMPA receptor potentiator.

2. The method of claim 1 wherein the first component is chosen from fluoxetine, duloxetine, venlafaxine, milnacipran, citalopram, fluvoxamine, paroxetine, sertraline, reboxetine, imipramine, rolipram, and buproprion.

3. The method of claim 2 wherein the first component is fluoxetine.

4. The method of claim 1 wherein the second component is chosen from [(methylethyl)sulfonyl]{2-[4-(4-{2-[(methylsulfonyl)amino]ethyl}phenyl)phenyl]propyl}amine, {(2R)-2-[4-(4-{2-[(methylsulfonyl)amino]ethyl}phenyl)phenyl]propyl}[(methylethyl)sulfonyl]amine, N-2-(4-(3-thienyl)phenylpropyl 2-propanesulfonamide, [2-fluoro-2-(4-{3-[(methylsulfonyl)amino]phenyl}phenyl)propyl][(methylethyl)sulfonyl]amine, [2-fluoro-2-(4-{3-[(methylsulfonyl)amino]phenyl}phenyl)propyl][(methylethyl)sulfonyl]amine (enantiomer 1), and [2-fluoro-2-(4-{3-[(methylsulfonyl)amino]phenyl}phenyl)propyl][(methylethyl)sulfonyl]amine (enantiomer 2).

5. A method for treating depression, comprising administering to a patient an effective amount of fluoxetine, in combination with an effective amount of {(2R)-2-[4-(4-{2-[(methylsulfonyl)amino]ethyl}phenyl)phenyl]propyl}[(methylethyl)sulfonyl]amine.

6. A pharmaceutical composition which comprises a first component which is a suitable antidepressant, and a second component which is a suitable AMPA receptor potentiator, the two components being present in an amount effective in the treatment of depression.

7. A composition of claim 6 which comprises a first component chosen from fluoxetine, duloxetine, venlafaxine, milnacipran, citalopram, fluvoxamine, paroxetine, sertraline, reboxetine, imipramine, rolipram, and buproprion, in combination with a second component chosen from [(methylethyl)sulfonyl]{2-[4-(4-{2-[(methylsulfonyl)amino]ethyl}phenyl)phenyl]propyl}amine, {(2R)-2-[4-(4-{2-[(methylsulfonyl)amino]ethyl}phenyl)phenyl]propyl}[(methylethyl)sulfonyl]amine, N-2-(4-(3-thienyl)phenylpropyl 2-propanesulfonamide, [2-fluoro-2-(4-{3-[(methylsulfonyl)amino]phenyl}phenyl)propyl][(methylethyl)sulfonyl]amine, [2-fluoro-2-(4-{3-[(methylsulfonyl)amino]phenyl}phenyl)propyl][(methylethyl)sulfonyl]amine (enantiomer 1), and [2-fluoro-2-(4-{3-[(methylsulfonyl)amino]phenyl}phenyl)propyl][(methylethyl)sulfonyl]amine (enantiomer 2).

8. A composition of claim 7 which is adapted for oral administration.

9. A pharmaceutical composition which comprises fluoxetine and {(2R)-2-[4-(4-{2-[(methylsulfonyl)amino]ethyl}phenyl)phenyl]propyl}[(methylethyl)sulfonyl]amine.

10. A pharmaceutical composition which comprises imipramine and {(2R)-2-[4-(4-{2-[(methylsulfonyl)amino]ethyl}phenyl)phenyl]propyl}[(methylethyl)sulfonyl]amine.

11. The use of an effective amount of a first component which is a suitable antidepressant, in combination with an effective amount of a second component which is a suitable AMPA receptor potentiator, for the manufacture of a medicament for the treatment of depression.

12. The use according to claim 11 wherein the first component is chosen from fluoxetine, duloxetine, venlafaxine, milnacipran, citalopram, fluvoxamine, paroxetine, sertraline, reboxetine, imipramine, rolipram, and buproprion.

13. The use according to claim 12 wherein the first component is fluoxetine.

14. The use according to any one of claims 11 to 13 wherein the second component is chosen from [(methylethyl)sulfonyl]{2-[4-(4-{2-[(methylsulfonyl)amino]ethyl}phenyl)phenyl]propyl}amine, {(2R)-2-[4-(4-{2-[(methylsulfonyl)amino]ethyl}phenyl)phenyl]propyl}[(methylethyl)sulfonyl]amine, N-2-(4-(3-thienyl)phenylpropyl 2-propanesulfonamide, [2-fluoro-2-(4-{3-[(methylsulfonyl)amino]phenyl}phenyl)propyl][(methylethyl)sulfonyl]amine, [2-fluoro-2-(4-{3-[(methylsulfonyl)amino]phenyl}phenyl)propyl][(methylethyl)sulfonyl]amine (enantiomer 1), and [2-fluoro-2-(4-{3-[(methylsulfonyl)amino]phenyl}phenyl)propyl][(methylethyl)sulfonyl]amine (enantiomer 2).

15. An article of manufacture comprising packaging material and a pharmaceutical composition which comprises a first component which is a suitable antidepressant, and a second component which is a suitable AMPA receptor potentiator, contained within said packaging material, wherein said packaging material comprises a label which indicates that said pharmaceutical composition can be used for treating depression.
